(19) United States
(12) Patent Application Publication
(10) Pub. No.: US 2021/0213049 A1
(43) Pub. Date: Jul. 15, 2021

(54) METHOD AND MOLECULAR HYDROGEN-CONTAINING COMPOSITION FOR PROMOTION OF POSTOPERATIVE RECOVERY

(71) Applicant: MiZ Company Limited, Kamakura-shi (JP)

(72) Inventors: Fumitake SATOH, Kanagawa (JP); Hideyuki SASAKI, Tokyo (JP); Ryosuke KUROKAWA, Kanagawa (JP); Shinichi HIRANO, Kanagawa (JP); Yusuke ICHIKAWA, Kanagawa (JP)

(21) Appl. No.: 17/147,692

(22) Filed: Jan. 13, 2021

(30) Foreign Application Priority Data
Jan. 15, 2020 (JP) 2020-004154

(51) Int. Cl. A61K 33/00; A61K 45/06; A61P 43/00

(52) U.S. Cl. CPC A61K 33/00; A61P 43/00; A61K 45/06

(57) ABSTRACT

The present invention provides a method or a composition for promotion of improvement of postoperative recovery. More specifically, the present invention provides a composition for promotion of recovery from or improvement of surgical invasion and/or a symptom associated with a surgery in a subject undergoing the surgery, comprising molecular hydrogen as an active ingredient, and a method for promotion of recovery from or improvement of surgical invasion and/or a symptom associated with a surgery in a subject undergoing the surgery, comprising administering a composition comprising molecular hydrogen as an active ingredient to the subject.

### RELATED APPLICATIONS

This application claims priority to Japanese Patent Application No. 2020-004154, filed on Jan. 15, 2020, the entire content of which is incorporated herein by reference.

### BACKGROUND OF THE INVENTION

### 1. Field of the Invention

The present invention provides a method or a molecular hydrogen-containing composition for promotion of recovery from or improvement of surgical invasion and a symptom in a subject.

### 2. Description of the Related Art

A variety of surgeries are performed in clinical settings. Surgeries are invasive and require care and management of hemorrhage, infection, pain, and the like. Post-operative follow-up is performed to monitor good recovery.

Pain is often experienced after anesthesia disappears, depending on the surgery, and measures such as administration of analgesics are taken to relieve such pain. However, adequate measures cannot be deemed to be taken to promote postoperative recovery. A search of patent literature has found that there are very few documents on promotion of postoperative recovery, apart from Japanese Translation of PCT International Patent Application No. 2017-513882.

Japanese Translation of PCT International Patent Application No. 2017-513882 discloses a method for improving and/or promoting recovery from hip fracture surgery by using a composition comprising a myostatin antagonist.

Since hydrogen, the active ingredient of the present invention, has anti-oxidative reactivity, which reduces oxidative stress caused by reactive oxygen species, the possibility has been pointed out that hydrogen can be used for the treatment of various diseases associated with oxidative stress (Ohta, S., Journal of Japanese Biochemical Society, 87(1):82-90, 2015). However, there are few cases for which clinical effectiveness in humans has actually been documented.

Promotion of postoperative (being also referred to as “after surgery”) recovery would make it possible not only to achieve relief of distress and improvement in quality of life of patients, but also to contribute to reduction in healthcare costs. As described above, few components or substances are known to be useful for promotion of postoperative recovery.

Under such circumstances, an object of the present invention is to promote postoperative recovery by using molecular hydrogen and/or to promote improvement of a symptom associated with a surgery.

### SUMMARY OF THE INVENTION

That is, the present invention encompasses the following characteristics:

(1) A composition for promotion of recovery from or improvement of surgical invasion and/or a symptom associated with a surgery in a subject undergoing the surgery, comprising molecular hydrogen as an active ingredient.

(2) The composition according to (1), which is a liquid or a gas comprising the molecular hydrogen.

(3) The composition according to (2), wherein the liquid comprising the molecular hydrogen has a hydrogen concentration of 1 to 10 ppm.

(4) The composition according to (2), wherein the gas comprising the molecular hydrogen has a hydrogen concentration of higher than zero (0) and not higher than 18.5% by volume.

(5) The composition according to any of (1) to (4), wherein the subject is a mammalian including a human.

(6) The composition according to any of (1) to (5), which is produced by using a hydrogen gas generating apparatus, a hydrogen water generating apparatus, or a hydrogen gas adding apparatus.

(7) The composition according to any of (1) to (6), wherein the surgical invasion is a stimulus selected from the group consisting of wound, pain, infection, hemorrhage, and inflammation that are caused by the surgery of the subject and disrupt homeostasis in an internal environment of the body.

(8) The composition according to any of (1) to (7), wherein the symptom associated with a surgery is a symptom selected from the group consisting of symptoms such as pain, hemorrhage, inflammation, pyrexia, muscle stiffness, numbness, edema, and convulsion that occur after the surgery performed for treatment of a disease, lesion, or disorder of the subject, and/or symptoms caused by the disease, lesion, or disorder of the subject.

### DETAILED DESCRIPTION OF THE PREFERRED EMBODIMENTS

The present invention can promote recovery from or improvement of surgical invasion and/or a symptom associated with a surgery in a subject undergoing the surgery.

The present invention will be described in more detail below.

1. Composition for Promotion of Postoperative Recovery

The present invention provides a composition comprising molecular hydrogen as an active ingredient which promotes recovery from or improvement of surgical invasion and/or symptoms associated with surgery in subjects undergoing the surgery.

In the present specification, the term “surgical invasion” includes, regardless of the type of a surgery performed for a subject, stimuli that are caused by the surgery and disrupt homeostasis in the internal environment of the body, such as wound, pain, infection, hemorrhage, and inflammation. A surgery is performed to eliminate the cause of a disease or a disorder and to recover and improve function of the site of the disease or the disorder. Examples of the surgery include resection of a lesion and orthopedic surgery or transplantation of an organ or a tissue. Usually, a surgery is invasive because it damages the body. There are a wide variety of surgeries, including open abdominal/chest surgery, which is highly invasive, endoscopic surgery, which is minimally invasive (e.g., polypectomy, laparoscopic surgery, robotic surgery), and surgery of skin/breast, muscles/bones, respiratory organ/chest, circulatory organ/spleen, digestive organs, urinary/genital organs, endocrine organs, sensory organs/visual organ auditory organ, malignant neoplasm, or the like.

More specifically, the term “surgery” has the meaning of the term generally used in the medical field and the healthcare industry and encompasses, but is not limited to, all surgeries caused by diseases, disorders, accidents, and the like that require surgery. Examples thereof include resection of benign or malignant tumors (e.g., cancer, fibroid), hypertrophic organs (e.g., hypertrophy of the prostate or the tonsilla), the appendix (appendicitis), or the like, surgery of organs such as the kidneys, liver, large intestine, stomach, lungs, heart, or breasts, surgery for fracture, bone deformation, disc hernia, arthropathy, or the like, transplantation of organs, cornea, skin, or the like, surgery for implantation of artifacts such as artificial bones or artificial joints, surgery for cerebral infarction, subarachnoid hemorrhage, brain contusion, or the like, surgery for burn wound, lacerated wound, contusion, or the like, and surgery in the ophthalmology field for cataract, retinal detachment, or the like.

In the present specification, the term “symptom associated with a surgery” includes symptoms (e.g., pain, hemorrhage, inflammation, pyrexia, muscle stiffness, numbness, edema, convulsion) that occur after surgery performed for the treatment of a disease (or a lesion) or a disorder and/or symptoms caused by a disease (or a lesion) or a disorder. For example, patients with coxarthrosis who have undergone hip replacement surgery to implant an artificial hip joint often experience symptoms such as muscle stiffness, pain, and numbness.

In the present specification, the term “subject” includes mammalians such as primates including humans, pet animals such as dogs and cats, and ornamental animals such as zoo animals. Preferred subjects are humans.

In the present specification, “hydrogen,” the active ingredient of the composition of the present invention, is molecular hydrogen (i.e., gaseous hydrogen or hydrogen gas) and is simply referred to as “hydrogen” or “hydrogen gas” unless otherwise specified. Additionally, the term “hydrogen” used in the present specification refers to a molecular formula of H₂, D₂ (deuterium), or HD (deuterated hydrogen) or a gas mixture thereof. D₂ is expensive but known to have a stronger superoxide eliminating effect than that of H₂. Hydrogen that can be used in the present invention is H₂, D₂ (deuterium), HD (deuterated hydrogen), or a gas mixture thereof, preferably H₂. Alternatively, D₂, and/or HD can be used instead of H₂ or in a mixture with H₂.

Preferred embodiments of the composition of the present invention are gases or liquids containing molecular hydrogen, preferably gases containing molecular hydrogen.

The gases containing molecular hydrogen are preferably air containing hydrogen gas or a mixed gas containing hydrogen gas and oxygen gas. The concentration of hydrogen gas in a gas containing molecular hydrogen (i.e., the composition of the present invention) is higher than zero (0) and not higher than 18.5% by volume, for example, 0.5% to 18.5% by volume, preferably 1% to 10% by volume, for example, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, and the like. In the present invention, higher hydrogen gas concentrations (but below the explosion limit) or higher daily hydrogen doses tend to be associated with greater effects of promoting postoperative recovery or improvement (e.g., suppression or alleviation) of symptoms.

Because hydrogen is a flammable and explosive gas, it is preferable to add hydrogen to the composition of the present invention under conditions safe for subjects such as humans and administer the mixture to subjects to promote postoperative recovery.

When a gas other than hydrogen gas is air, the air concentration is in the range of, for example, 81.5% to 99.5% by volume.

When a gas other than hydrogen gas is a gas containing oxygen gas, the oxygen gas concentration is in the rage of, for example, 21% to 99.5% by volume.

As another main gas, for example, nitrogen gas can be further added.

The liquids containing molecular hydrogen are specifically aqueous liquids containing a dissolved hydrogen gas. Examples of the aqueous liquids used herein include, but are not limited to, water (e.g., purified water, sterilized water), physiological saline, buffer solutions (e.g., buffer solutions of pH 4 to 7.4), drip infusion solutions, fluid infusion solutions, injection solutions, and drinks (e.g., tea drinks such as green tea and black tea, fruit juice, green juice, vegetable juice). Examples of the hydrogen concentration in a liquid containing molecular hydrogen include, but are not limited to, 1 to 10 ppm, preferably 1,2 to 9 ppm, for example, 1,5 to 9 ppm, 1.5 to 8 ppm, 1.5 to 7 ppm, 1,5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, and 5 to 7 ppm.

In the present invention, higher concentrations of dissolved hydrogen (but below the explosion limit) or higher daily hydrogen doses tend to be associated with greater effects of promoting postoperative recovery.

A gas or a liquid containing molecular hydrogen is formulated to provide a predetermined hydrogen gas concentration and then with the same, for example, a pressure-resistant container (e.g., a stainless cylinder, an aluminum can, a pressure-resistant plastic bottle [e.g., a pressure-resistant PET bottle] and a plastic bag preferably having the inside laminated with an aluminum film, or an aluminum bag) is filled. Aluminum has the property of unlikely allowing hydrogen molecules to pass therethrough. Alternatively, a gas containing molecular hydrogen or a liquid containing molecular hydrogen may be produced in situ before use by using an apparatus such as a hydrogen gas generating apparatus, a hydrogen water generating apparatus, or a hydrogen gas adding apparatus such as a known or commercially available hydrogen gas supply apparatus (an apparatus for generating a gas containing molecular hydrogen), a hydrogen adding device (an apparatus for hydrogen water generation), or a non-destructive hydrogen adding apparatus (e.g., an apparatus for non-destructively adding hydrogen gas into a bag for a biocompatible solution such as a drip infusion solution).

The hydrogen gas supply apparatus enables hydrogen gas generated from a reaction of a hydrogen generating agent (e.g., metallic aluminum, magnesium hydride) and water to be mixed with a diluent gas (e.g., air, oxygen) in a predetermined ratio (refer to Japanese Patent No. 5228142, etc.). Or, the hydrogen gas supply apparatus mixes hydrogen gas generated utilizing electrolysis of water with a diluent gas such as oxygen or air (refer to Japanese Patent No. 5502973, Japanese Patent No. 5900688, etc.). Thus, a gas containing molecular hydrogen at a hydrogen concentration in the range of, for example, 0.5% to 18.5% by volume can be prepared.

The hydrogen adding device is an apparatus that generates hydrogen by using a hydrogen generating agent and a pH modifier and dissolving the hydrogen in a biocompatible solution such as water (refer to Japanese Patent No. 4756102, Japanese Patent No. 4652479, Japanese Patent No. 4950352, Japanese Patent No. 6159462, Japanese Patent No. 6170605, Japanese Patent Laid-open No. 2017-104842, etc.). Examples of a mixture of a hydrogen generating agent and a pH modifier include metallic magnesium and a strongly acidic ion exchange resin or an organic acid (e.g., malic acid, citric acid) and a metallic aluminum powder and a calcium hydroxide powder. With these mixtures, a liquid containing molecular hydrogen at a dissolved hydrogen concentration of, for example, approximately 1 to 10 ppm can be prepared.

The non-destructive hydrogen adding apparatus is an apparatus or a device that adds hydrogen gas to a commercially available biocompatible solution such as a drip infusion solution (e.g., enclosed in a hydrogen-permeable plastic bag such as a polyethylene bag) from the outside of a package and is commercially available from, for example, MiZ Company Limited (http://www.e-miz.co.jp/technology.html). This apparatus can dissolve hydrogen in a biocompatible solution aseptically until the equilibrium concentration is reached, by immersing a bag containing the biocompatible solution in saturated hydrogen water, so that hydrogen is permeated into the bag. The apparatus is composed of, for example, an electrolytic bath and a water bath, and water in the water bath is circulated in the electrolytic bath and the water bath to generate hydrogen by electrolysis. Or, a simplified, disposable device can be used for a similar purpose (refer to Japanese Patent Laid-open No. 2016-112562, etc.). This device has a biocompatible solution-containing plastic bag (a hydrogen-permeable bag, for example, a polyethylene bag) and a hydrogen generating agent (e.g., metallic calcium, metallic magnesium/cation exchange resin) incorporated in an aluminum bag, and the hydrogen generating agent is wrapped with, for example, a non-woven fabric (e.g., steam-permeable non-woven fabric). Hydrogen generated by wetting the hydrogen generating agent wrapped with a non-woven fabric with a small amount of water, such as a steam, is dissolved in a biocompatible solution non-destructively and aseptically.

Or, a purified hydrogen gas cylinder, a purified oxygen gas cylinder, or a purified air cylinder may be provided to produce a gas or a liquid containing molecular hydrogen which is adjusted to provide a predetermined hydrogen concentration or a predetermined oxygen or air concentration.

A gas containing molecular hydrogen or a liquid containing molecular hydrogen (e.g., water [e.g., purified water, sterilized water], physiological saline, drip infusion solution) prepared using the above-mentioned apparatuses or devices can be administered orally or parenterally to subjects undergoing surgery preoperatively, perioperatively, or postoperatively.

Other embodiments of the composition of the present invention include dosage forms (e.g., tablets, capsules) prepared to be orally administered to (or ingested by) subjects, which contain a hydrogen generating agent that enables hydrogen to be generated in the gastrointestinal tract. The hydrogen generating agent preferably comprises, for example, components approved as food or food additives.

When the composition of the present invention comprises molecular hydrogen as an active ingredient, examples of the method of administering the composition to subjects include administration by inhalation, suction or the like. For example, transpulmonary administration is preferred. When a liquid containing molecular hydrogen is contained as an active ingredient, oral or intravenous administration (including drip infusion) is preferred. When a gas is inhaled, the gas is inhaled from the mouth or the nose via a nasal cannula or a mask-like device covering the mouth and the nose, transported to the lungs, and delivered to the whole body by blood.

The liquid containing molecular hydrogen to be orally administered may be administered to subjects as a cooled liquid or a liquid stored at room temperature. Hydrogen is dissolved in water at a concentration of approximately 1.6 ppm (1.6 mg/L) at room temperature and under a normal pressure, and the difference in solubility due to temperature is known to be relatively small. Or, when a liquid containing molecular hydrogen is, for example, in the form of a drip infusion solution or an injection solution containing hydrogen gas prepared using the above-described non-destructive hydrogen adding apparatus, the liquid may be administered to subjects by parenteral routes, such as intravenous or intraarterial administration.

One dose or multiple doses (e.g., two to three doses) per day of a gas containing molecular hydrogen at the above-mentioned hydrogen concentrations or a liquid containing molecular hydrogen at the above-mentioned dissolved hydrogen concentrations can be administered to humans for a period of one week to three months or longer, for example, one week to six months or longer (e.g., one year or longer, two years or longer). When a gas containing molecular hydrogen is administered, the gas is preferably inhaled for at least 30 minutes per dose. Because the improving effect becomes higher with a longer inhalation time, the gas can be administered for, for example, 30 minutes to one hour, one hour to two hours, two hours to three hours, or longer. Additionally, when a gas containing molecular hydrogen is administered in a transpulmonary manner by inhalation or suction, the gas can be administered to subjects under an atmospheric pressure environment, or, for example, under a high atmospheric pressure in the range exceeding a standard atmospheric pressure (i.e., approximately 1.013 atm) and not higher than 7.0 atm, for example, under a high atmospheric pressure environment in the range of 1.02 to 7.0 atm, preferably in the range of 1.02 to 5.0 atm, more preferably in the range of 1.02 to 4.0 atm, yet more preferably in the range of 1.02 to 1.35 atm (including the gas containing molecular hydrogen).

2. A Method for Promoting Postoperative Recovery

The present invention further provides a method for promoting recovery from or improvement of surgical invasion and/or a symptom associated with a surgery in a subject undergoing the surgery, using the above-mentioned composition comprising molecular hydrogen as an active ingredient. The composition is as defined or mentioned in section 1 above and includes all embodiments therein.

The composition containing molecular hydrogen, surgical invasion, symptom associated with a surgery, dose, administration method, and the like are as described in the above 1.

In the method of the present invention, the composition of the present invention can be administered to a subject preoperatively, perioperatively, or postoperatively, for example, from preoperative days 1 to 7 or from immediately after surgery.

In the method of the present invention, a gas containing molecular hydrogen (preferably, air or oxygen) at higher than zero (0) and not higher than 18.5% by volume, for example, 0.5% to 18.5% by volume, 2% to 10% by volume, 2% to 9% by volume, 2% to 8% by volume, 3% to 10% by volume, 3% to 9% by volume, 3% to 8% by volume, 3% to 7% by volume, 3% to 6% by volume, 4% to 10% by volume, 4% to 9% by volume, 4% to 8% by volume, 4% to 7% by volume, 4% to 6% by volume, 4% to 5% by volume, 5% to 10% by volume, 5% to 9% by volume, 5% to 8% by volume, 6% to 10% by volume, 6% to 9% by volume, 6% to 8% by volume, 6% to 7% by volume, or the like, preferably 5% to 10% by volume, 5% to 8% by volume, for example, 6% to 10% by volume, 6% to 8% by volume, 6% to 7% by volume, or the like can be inhaled or sucked by subjects for, for example, one to three hours or longer per day and can be continued for, for example, one to three months or longer, four to seven months or longer, one to three years or longer.

Or, in the method of the present invention, for example, 200 to 500 mL per dose for intravenous administration or, for example, 500 to 1000 mL per dose for oral administration of a liquid containing molecular hydrogen at a concentration of, for example, 1 to 10 ppm, 1.5 to 9 ppm, 1.5 to 8 ppm, 1.5 to 7 ppm, 1.5 to 6 ppm, 1.5 to 5 ppm, 1.5 to 4 ppm, 2 to 10 ppm, 2 to 9 ppm, 2 to 8 ppm, 2 to 7 ppm, 2 to 6 ppm, 2 to 5 ppm, 3 to 10 ppm, 3 to 9 ppm, 3 to 8 ppm, 3 to 7 ppm, 4 to 10 ppm, 4 to 9 ppm, 4 to 8 ppm, 4 to 7 ppm, 5 to 10 ppm, 5 to 9 ppm, 5 to 8 ppm, 5 to 7 ppm, or the like, preferably 3 to 10 ppm, 4 to 10 ppm, 5 to 10 ppm, 5 to 9 ppm, 5 to 8 ppm, 5 to 7 ppm, or the like can continue to be administered to subjects for, for example, 0.5 to three months or longer, four to seven months or longer, one to three years or longer.

The method of the present invention may further be used in combination with a therapy such as chemotherapy, radiation therapy, or rehabilitation, if necessary. Such a combination use is expected to increase the effect of promoting postoperative recovery/improvement.

Rehabilitation is performed to recover the body's reduced function after surgery. For example, rehabilitation is thought to be essential for elderly patients because they are likely to have a markedly reduced physical function due to postoperative hospitalization. Specifically, rehabilitation is performed with a program for recovery of physical function, comprising an appropriate combination of physiotherapy, occupational therapy, speech-language-hearing therapy, counseling to address social and emotional needs, and the like.

Additionally, the method of the present invention is preferably used in combination with chemotherapy for symptoms such as pain. Examples of analgesics include Voltaren, Loxonin, and morphine.

The method of the present invention can include the following characteristics.

(1′) A method for promotion of recovery from or improvement of surgical invasion and/or a symptom associated with a surgery in a subject undergoing the surgery, comprising administering a composition comprising molecular hydrogen as an active ingredient to the subject.

(2′) The method of (1′), wherein the composition is a liquid or a gas comprising the molecular hydrogen.

(3′) The method of (2′), wherein the liquid comprising the molecular hydrogen has a hydrogen concentration of 1 to 10 ppm.

(4′) The method of (2′), wherein the gas comprising the molecular hydrogen has a hydrogen concentration of higher than zero (0) and not higher than 18.5% by volume.

(5′) The method of (1′), wherein the subject is a mammalian including a human,

(6′) The method of (1′), wherein the composition is produced by using a hydrogen gas generating apparatus, a hydrogen water generating apparatus, or a hydrogen gas adding apparatus.

(7′) The method of (1′), wherein the surgical invasion is a stimulus selected from the group consisting of wound, pain, infection, hemorrhage, and inflammation that are caused by the surgery of the subject and disrupt homeostasis in an internal environment of the body.

(8′) The method of (1′), wherein the symptom associated with a surgery is a symptom selected from the group consisting of symptoms such as pain, hemorrhage, inflammation, pyrexia, muscle stiffness, numbness, edema, and convulsion that occur after the surgery performed for treatment of a disease, lesion, or disorder of the subject, and/or symptoms caused by the disease, lesion, or disorder of the subject.

(9′) The method of (1′), wherein the composition is administered to the subject preoperatively, perioperatively, or postoperatively.

(10′) The method of (9′), wherein the composition is administered to the subject from preoperative days 1 to 7 or from immediately after surgery.

(11′) The method of (1′), which is performed optionally in combination with a therapy including chemotherapy, radiation therapy, or rehabilitation, thereby to increase an effect of promoting postoperative recovery/improvement.

### EXAMPLE

The present invention is explained more specifically with reference to the following example. However, the example is not intended to limit the scope of the present invention.

### Example 1

<A Case of Early Postoperative Recovery by Hydrogen Inhalation>

A 65-year-old man underwent hip replacement surgery because of coxarthrosis (his right leg being 41.5 cm shorter). The surgery was initiated at 10:00 a.m. on Jul. 10, 2019 and completed around 11 o'clock a.m. on the same day. Immediately after waking up from the surgery, the patient initiated inhalation of a hydrogen gas in the patient's wardroom. Because the patient's leg was swollen with pulled and rigid muscles after completion of the surgery, the patient initiated rehabilitation simultaneously. From immediately after awakening, the hydrogen gas was inhaled for three to four hours per day during the rehabilitation period using a hydrogen generating machine (type, Jobs-α; a hydrogen concentration, approximately 5.0%; generation rate of hydrogen gas, approximately 200 mL/min) manufactured by MiZ Company Limited.

Initially, use of crutches for three months was planned as in usual cases, based on the anticipated postoperative course (that is, the patient was expected to use both crutches for the first 1.5 months and one crutch for the subsequent 1.5 months). However, hydrogen inhalation from immediately after the surgery achieved early postoperative recovery, and the patient was discharged from hospital on July 20. He did not need to use crutches on Jul. 23, 2019 (at 13 days after the surgery), and the patient's state of recovery surprised the staff in charge of rehabilitation.

### INDUSTRIAL APPLICABILITY

The present invention can promote postoperative recovery and improve a symptom by administering a composition containing molecular hydrogen.

All the publications, patents, and patent applications cited herein are incorporated herein by reference in their entirety.

What is claimed is:

1. A method for promotion of recovery from or improvement of surgical invasion and/or a symptom associated with a surgery in a subject undergoing the surgery, comprising administering a composition comprising molecular hydrogen as an active ingredient to the subject.

2. The method of claim 1, wherein the composition is a liquid or a gas comprising the molecular hydrogen.

3. The method of claim 2, wherein the liquid comprising the molecular hydrogen has a hydrogen concentration of 1 to 10 ppm.

4. The method of claim 2, wherein the gas comprising the molecular hydrogen has a hydrogen concentration of higher than zero (0) and not higher than 18.5% by volume.

5. The method of claim 1, wherein the subject is a mammalian including a human.

6. The method of claim 1, wherein the composition is produced by using a hydrogen gas generating apparatus, a hydrogen water generating apparatus, or a hydrogen gas adding apparatus.

7. The method of claim 1, wherein the surgical invasion is a stimulus selected from the group consisting of wound, pain, infection, hemorrhage, and inflammation that are caused by the surgery of the subject and disrupt homeostasis in an internal environment of the body.

8. The method of claim 1, wherein the symptom associated with a surgery is a symptom selected from the group consisting of symptoms such as pain, hemorrhage, inflammation, pyrexia, muscle stiffness, numbness, edema, and convulsion that occur after the surgery performed for treatment of a disease, lesion, or disorder of the subject, and/or symptoms caused by the disease, lesion, or disorder of the subject.

9. The method of claim 1, wherein the composition is administered to the subject preoperatively, perioperatively, or postoperatively.

10. The method of claim 9, wherein the composition is administered to the subject from preoperative days 1 to 7 or from immediately after surgery.

11. The method of claim 1, which is performed optionally in combination with a therapy including chemotherapy, radiation therapy, or rehabilitation, thereby to increase an effect of promoting postoperative recovery/improvement.
